(19) United States
(12) Patent Application Publication
(10) Pub. No.: US 2010/0208203 A1
(43) Pub. Date: Aug. 19, 2010

(54) Compact ocular wavefront system with long working distance

(76) Inventor: Edwin J. Sarver, Carbondale, IL (US)

Correspondence Address: MCHALE & SLAVIN, P.A., 2855 PGA BLVD, PALM BEACH GARDENS, FL, 33410, US

(21) Appl. No.: 12/708,047

(22) Filed: Feb. 18, 2010

Related U.S. Application Data
(60) Provisional application No. 61/153,532, filed on Feb. 18, 2009.

(51) Int. Cl. A61B 3/14; A61B 3/10

(52) U.S. Cl. 351/206; 351/205

(57) ABSTRACT

A compact ocular wavefront system with a long working distance is disclosed for use in reducing the overall optical path length for an ocular wavefront system while providing performance similar to that of a traditional system. The system incorporates a compact three-lens subsystem to relay the wavefront from the eye's pupil to a wavefront sensor. The wavefront sensor is placed in close proximity to a digital camera's sensor array. The combination of the compact relay system and the location of the wavefront sensor allows the total track of a traditional ocular wavefront system to be reduced significantly.

### CROSS REFERENCE TO RELATED APPLICATIONS

This application is based upon, and claims the priority date of, U.S. Provisional Application No. 61/153,532 filed Feb. 18, 2009 entitled “Compact Ocular Wavefront System with Long Working Distance”, the contents of which are incorporated herein by reference.

### FIELD OF THE INVENTION

This invention is directed to ocular wavefront systems, and more particular, to a method and device for measuring the aberrations of the human eye.

### BACKGROUND OF THE INVENTION

Ocular wavefront systems are used to measure the aberrations of the human eye. These aberrations include the lower order aberrations (sphere, cylinder, and axis) such as those used to correct vision using spectacles or contact lenses, and higher order aberrations used to correct additional vision defects such as spherical aberrations or coma. These systems have found wide use for planning and evaluating refractive surgery techniques such as LASIK.

A typical ocular wavefront system is shown in FIG. 1. In this system, light from a super luminscent diode (SLD) is partially reflected by beam splitter (BS) into the eye of a subject being measured. The light forms a diffuse reflection at the retina of the eye. The light from the diffuse reflection exits the eye and is relayed by lenses L1 and L2 onto sensor (S). The image from sensor S is brought into focus on the camera sensor via L3. Typical lens values are L1=L2=100 mm, L3=25 mm. The typical operation of the relay lens L1 and L2 is illustrated in FIG. 2 for a wavefront, and in FIG. 3 for the pupil boundary.

FIG. 2 is a ray tracing of the relay of the wavefront from the eye to the sensor. In this figure the wavefront is shown to be a plane wave. FIG. 3 is a ray tracing of the relay of the pupil boundary from the eye to the sensor plane. In FIG. 2, we show a plane wave leaving the eye's pupil. When it is refracted by lens L1 the rays are brought to a focus at the focal point of lens L1 at the focal distance F1 of lens L1. These focused rays then propagate to lens L2 a focal distance F2 downstream. At L2 the rays are again made parallel. In this way the wavefront from the eye's pupil is relayed to the sensor plane S. In FIG. 3, we show how the eye's pupil boundary is relayed to the sensor plane S. Since the pupil is at the focal distance F1 from lens L1, the rays refracted from L1 are parallel. They remain parallel until they reach L2 where they are brought into focus at the focal distance F2 of the lens. This focal distance is where the sensor S is located. We refer to the distance from the eye's pupil to the lens L1 as the working distance. This distance is often a fixed parameter for the system. We will refer to the distance from the lens L1 to the sensor S as the total track (T) of the relay lens. In the typical case where F1 and F2 are 100 mm, the working distance is 100 mm and the total track is 300 mm.

The sensor in most commercial ocular wavefront systems is a Hartmann-Shack micro lens array (two-dimensional array of equally spaced miniature lenses), a Hartmann-Screen (two-dimensional array of equally spaced apertures), or a pair of Ronchi grids (checker board patterns rotated with respect to each other). The sensor's focal plane is usually located fairly close to the sensor (on the order of a few mm). If a magnification of 1 is used from the sensor's focal plane to the camera sensor, the distance from the sensor plane to the camera sensor is about 100 mm, assuming a 25 mm focal length lens L3. Thus the total distance from lens L1 to the camera sensor plane is about 300+100=400 mm.

This 400 mm distance leads to a rather long optical path that must be properly housed in a commercial system. This leads to either a long enclosure or a folded system in which mirrors and/or prisms are used to re-direct the light path to make an enclosure dimension smaller. For most applications, a smaller optical path would provide advantages such as reduced cost, reduced weight, and increased convenience due to a decrease in overall enclosure size.

### SUMMARY OF THE INVENTION

Disclosed is a compact ocular wavefront system with a long working distance. The system incorporates a compact three-lens subsystem to relay the wavefront from the eye's pupil to a wavefront sensor. The wavefront sensor is placed in close proximity to a digital camera's sensor array. The combination of the compact relay system and the location of the wavefront sensor allows the total track of a traditional ocular wavefront system to be reduced significantly.

Thus, it is an objective of this invention to produce an ocular wavefront system with a significantly reduced optical path without reducing the overall optical performance of the system compared to the traditional relay system layout.

Another objective of this invention is to reduce the overall optical path length for an ocular wavefront system while providing performance similar to that of a traditional system.

Another objective of this invention is to provide a smaller optical path to provide advantages such as reduced cost, reduced weight, and increased convenience due to a decrease in overall enclosure size.

Still another objective of this invention is provide a compact relay system and positioning of a wavefront sensor to allow the total track of a traditional ocular wavefront system to be reduced significantly.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE DRAWINGS

FIG. 1 is an illustration of ray tracing of a typical ocular waverfront;

FIG. 2 is an illustration of ray tracing of a relay of wavefront from eye to sensor;

FIG. 3 is an illustration of ray tracing of a relay of a pupil boundary from eye to sensor plane;

FIG. 4 is an optical ray notation;

FIG. 5 is a propagation in homogeneous medium;

FIG. 6 is a refraction at an interface;

FIG. 7 is a two-lens system;

FIG. 8 is an illustration of a ray tracing of relay of wavefront from eye to sensor for the three-lens relay system;

FIG. 9 is an illustration of a ray tracing of relay of pupil boundary from eye to sensor plane for compact three-lens relay system; and

FIG. 10 is an illustration of a simplified optical system for compact ocular wavefront system having long working distance.

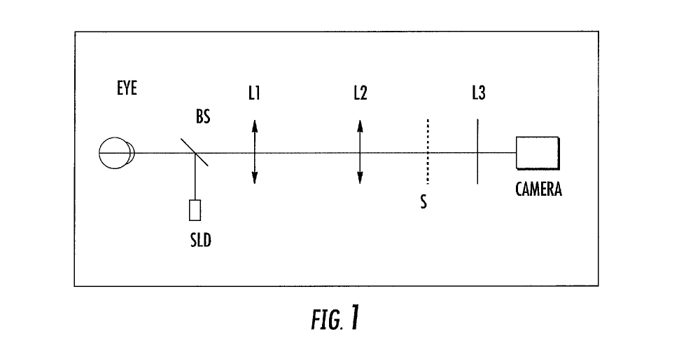

### DETAILED DESCRIPTION OF THE PREFERRED EMBODIMENT

In an effort to reduce the overall optical path length for an ocular wavefront system while providing performance similar to that of a traditional system, we perform two steps. First, we design a compact three-lens relay lens system that has the same properties as the original two-lens relay system L1 and L2. Second, we place the short focal length wavefront sensor at a focal distance away from the camera sensor. In the later case, we simply mount the sensor close to the camera sensor. In the former case, we wish to design a three-lens system of shorter total track T than the original two-lens relay system with the same optical system matrix S as the original relay system.

It is convenient to describe the paraxial properties of an optical system using the matrix optics formulation. Since there are various methods to develop matrix optics, we will explicitly state our system of equations. In our coordinate system, x is directed to the right along the optical axis, and y is directed up perpendicular to the optical axis.

### An Optical Ray

An optical ray is specified by its starting height and direction as shown in equation (1).

$$[ \begin{matrix}y \\ v\end{matrix} ] \qquad ( 1 )$$

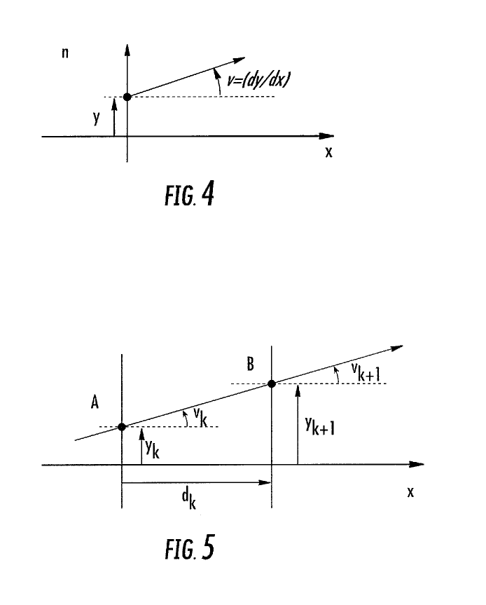

In this notation, an optical ray r is given by its height y and ray slope v. This is illustrated in FIG. 4. The ray slope v is defined as the change in y for a unit change in x or (dy/dx).

### Translation Matrix

The propagation of a ray in a homogeneous medium is illustrated in FIG. 5. In a homogeneous medium, a ray continues in a straight line. Thus, a ray starting at the plane A of height yₖ and direction vₖ will intersect plane B, located at a distance of dₖ from A, at height yₖ₊₁ given by

yₖ₊₁=yₖ+dₖ×vₖ

The direction remains unchanged so that

vₖ₊₁=vₖ

Combining these results in matrix form we have equation (2).

$$[ \begin{matrix}{y}_{k + 1} \\ {v}_{k + 1}\end{matrix} ] = [ \begin{matrix}1 & {d}_{k} \\ 0 & 1\end{matrix} ] \times [ \begin{matrix}{y}_{k} \\ {v}_{k}\end{matrix} ] \qquad ( 2 )$$

The translation matrix Tₖ is then identified as:

$$T = [ \begin{matrix}1 & {d}_{k} \\ 0 & 1\end{matrix} ] \qquad ( 3 )$$

### Refraction Matrix

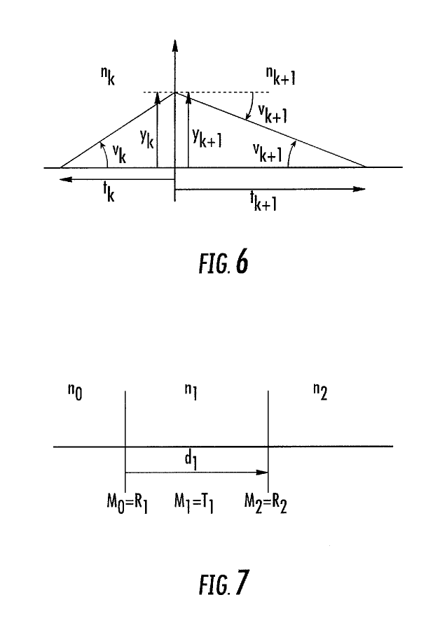

The refraction of a ray at the interface of two media of different index of refraction is illustrated in FIG. 6. As drawn in FIG. 6, vₖ is positive, yₖ is positive and tₖ is negative. At the interface where the power of the surface is P, by the paraxial approximation to Snell's law, we have the relations:

$$\frac{{n}_{k + 1}}{{t}_{k + 1}} = \frac{{n}_{k}}{{t}_{k}} + P$$

$${y}_{k + 1} = {y}_{k}$$

$$and$$

$${v}_{k} = - \frac{{y}_{k}}{{t}_{k}} {n}_{k}$$

$${v}_{k + 1} = - \frac{{y}_{k + 1}}{{t}_{k + 1}} {n}_{k + 1}$$

Combining these we have

$${v}_{k + 1} = \frac{{n}_{k}}{{n}_{k + 1}} {v}_{k} - \frac{P}{{n}_{k + 1}} {y}_{k}$$

Combining these results in matrix form we have equation (4).

$$[ \begin{matrix}{y}_{k + 1} \\ {v}_{k + 1}\end{matrix} ] = [ \begin{matrix}1 & 0 \\ - \frac{P}{{n}_{k + 1}} & \frac{{n}_{k}}{{n}_{k + 1}}\end{matrix} ] \times [ \begin{matrix}{y}_{k} \\ {v}_{k}\end{matrix} ] \qquad ( 4 )$$

The refraction matrix Rₖ is then identified as:

$$R = [ \begin{matrix}1 & 0 \\ - \frac{P}{{n}_{k + 1}} & \frac{{n}_{k}}{{n}_{k + 1}}\end{matrix} ] \qquad ( 5 )$$

### System Matrix

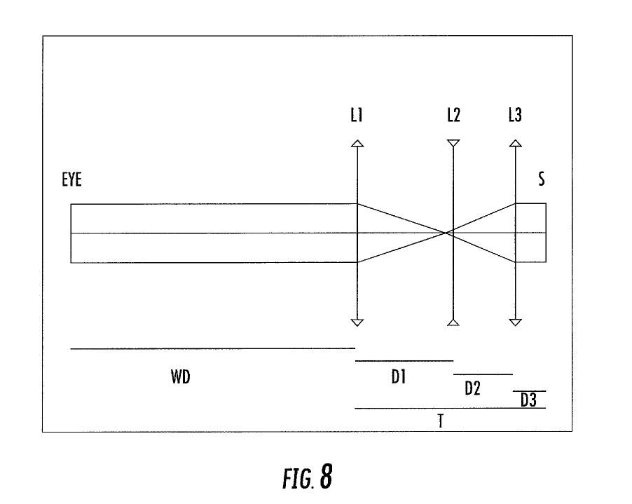

The translation and refraction matrices can be combined to compute how rays are transferred by a complete system. For example, consider the simple two lens system illustrated in FIG. 7. A ray r₀ incident at the first lens is refracted as follows to r₁:

r₁=M₀r₀

The intermediate ray r₁ refracted by the first lens is translated to be incident at the second lens as r₂:

$${\begin{matrix}{r}_{2} = {M}_{1} {r}_{1} \\ = {M}_{1} {M}_{0} r\end{matrix}}_{0}$$

The intermediate ray r₂ is refracted at the second lens and the resulting ray is r₃.

$$\begin{matrix}{r}_{3} = {M}_{2} {r}_{2} \\ = {M}_{2} {M}_{1} {r}_{1} \\ = {M}_{2} {M}_{1} {M}_{0} {r}_{0}\end{matrix}$$

The ray input to this simple optical system is r₀ and the output is r₃. We can represent the three matrices, R₁, T₁, and R₂, by a single matrix that represents how the ray r₀ is traced to the output r₃. This single matrix is referred to as the system matrix and for our simple two-lens case, the system matrix is:

S=R₂T₁R₁

Thus, the 2×2 system matrix S is computed by multiplying the individual translation and refraction matrices for the optical system. The system matrix for the original two-lens relay system (which we call the desired system matrix) is given by the product of translation and refraction matrices as shown in equation (6).

$$S = [ \begin{matrix}1 & F 2 \\ 0 & 1\end{matrix} ] \times [ \begin{matrix}1 & 0 \\ - 1 / F 2 & 1\end{matrix} ] \times [ \begin{matrix}1 & F 1 + F 2 \\ 0 & 1\end{matrix} ] \times [ \begin{matrix}1 & 0 \\ - 1 / F 1 & 1\end{matrix} ] \times [ \begin{matrix}1 & F 1 \\ 0 & 1\end{matrix} ] \qquad ( 6 )$$

Performing the multiplication and simplifying yields equation (7).

$$S = [ \begin{matrix}- F 2 / F 1 & F 2 \\ 0 & 1 - F 1 / F 2\end{matrix} ] \qquad ( 7 )$$

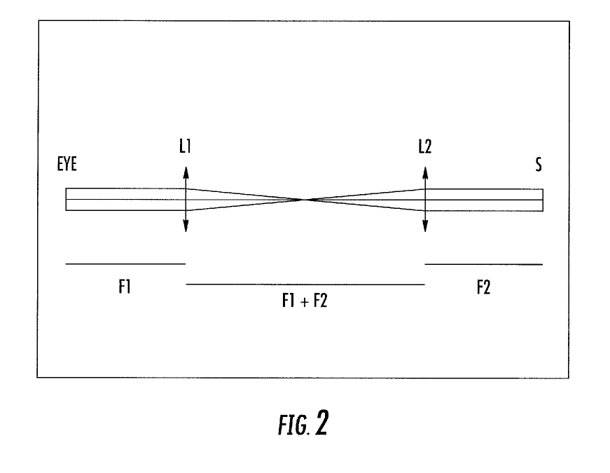

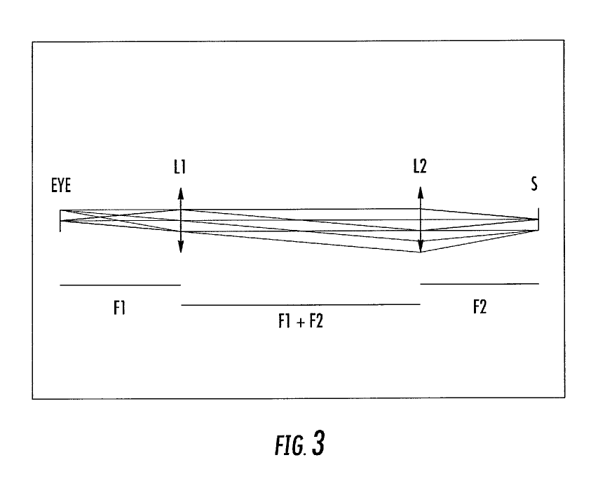

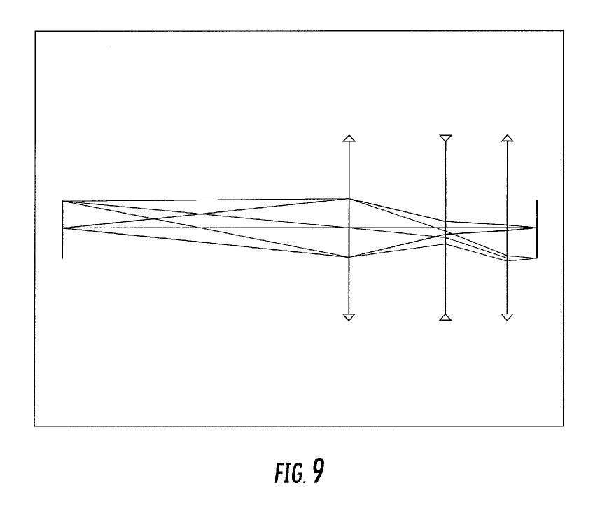

The compact three-lens relay system is shown in FIG. 8 depicting the ray tracing of relay of wavefront from eye to sensor for the three-lens relay system. In this figure, WD is the working distance and is the same as F1 from FIG. 2 for the original relay lens system. The distance D1 is the distance from L1 to L2, D2 is the distance from L2 to L3, and the D3 is the distance from L3 to the sensor plane S. The distance from the last lens L3 to the sensor plane S is much shorter than the distance F2 shown in FIG. 2. As noted above the total track T is the distance from the first lens L1 to the sensor plane.

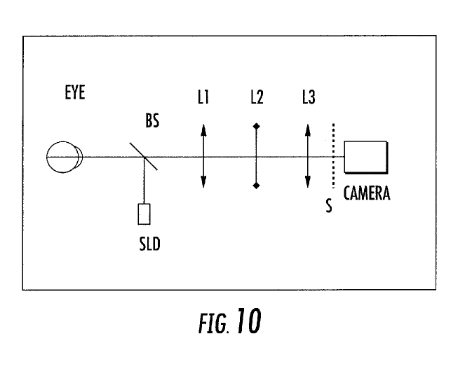

As shown in the diagram, L1 and L3 are converging (positive focal length) lenses while L2 is a diverging (negative focal length) lens. A ray tracing of the relaying of the pupil boundary to the sensor plane for the compact three-lens relay system is shown in FIG. 9. FIG. 9 is a ray tracing of relay of pupil boundary from eye to sensor plane for compact three-lens relay system.

The system matrix for the compact three-lens relay system (which we refer to as the current system matrix) is given by the product of translation and refraction matrices as shown in equation (8).

$$C = [ \begin{matrix}1 & D 3 \\ 0 & 1\end{matrix} ] \times [ \begin{matrix}1 & 0 \\ - 1 / F 3 & 1\end{matrix} ] \times [ \begin{matrix}1 & D 2 \\ 0 & 1\end{matrix} ] \times [ \begin{matrix}1 & 0 \\ - 1 / F 2 & 1\end{matrix} ] \times [ \begin{matrix}1 & D 1 \\ 0 & 1\end{matrix} ] \times [ \begin{matrix}1 & 0 \\ - 1 / F 1 & 1\end{matrix} ] \times [ \begin{matrix}1 & WD \\ 0 & 1\end{matrix} ] \qquad ( 8 )$$

Performing the multiplication and simplifying yields equation (9).

$$C = [ \begin{matrix}C 00 & C 01 \\ C 10 & C 11\end{matrix} ] \\ C 00 = \frac{D 1 \times D 2 \times F 3 - D 1 \times D 2 \times D 3 + D 1 \times D 3 \times F 3}{F {2}^{2} \times F 3} - \frac{\begin{matrix}D 1 \times F 3 - 2 \times D 2 \times D 3 - \\ D 1 \times D 3 + 2 \times D 2 \times F 3 + 2 \times D 3 \times F 3\end{matrix}}{F 2 \times F 3} - \frac{D 3 - F 3}{F 3} \\ C 01 = \frac{D 1 \times F 3 - D 2 \times D 3 - D 1 \times D 3 + D 2 \times F 3 + D 3 \times F 3}{F 3} - \frac{D 1 \times D 2 \times F 3 - D 1 \times D 2 \times D 3 + D 1 \times D 3 \times F 3}{F 2 \times F 3} \\ C 10 = \frac{D 1 + 2 \times D 2 - 2 \times F 3}{F 2 \times F 3} - \frac{1}{F 3} - \frac{D 1 \times D 2 - D 1 \times F 3}{F {2}^{2} \times F 3} \\ C 11 = \frac{D 1 \times D 2 - D 1 \times F 3}{F 2 \times F 3} - \frac{D 1 + D 2 - F 3}{F 3} \qquad ( 9 )$$

We define the error between the desired system matrix S and the current system matrix C as in equation (10).

$$E = {\sum }_{m = 0}^{1} {\sum }_{n = 0}^{1} {W}_{mn} \times {\vert {S}_{mn} - {C}_{mn} \vert}^{p} \qquad ( 10 )$$

In this equation, the error E is calculated using weights and corresponding elements from the S and C matrices. The weights Wmn allow individual terms in the system matrixes to receive more importance than others and the parameter p allows us to weight larger errors more or less than small errors. Generally, we find successful system parameters for the compact three-lens relay system setting all weights to 1 and the parameter p to 2.

The preferred calculation strategy is to be given a prototype two-lens relay system denoted by lenses La and Lb (we switch from our notation of use L1 and L2 for the two-lens system so as to avoid confusion with lenses L1, L2, and L3 for the three-lens relay system to be calculated), working distance WD=Fa, and select a desired total track T and sensor distance D3. We then calculate the prototype system matrix S using (2). Next, we use a global optimization algorithm (such as simulated annealing) to find the focal lengths F1, F2, and F3 and distances D1 and D2 so that the system matrix C from (4) equals the prototype system matrix S. It is not rigorously known if a solution is always possible, but experience has shown that a solution is usually found using simulated annealing for reasonable prototype system matrices S. As an example, for the case described above we have:
Fa=100, Fb=100
WD=100, T=65, D3=10

After optimization:
F1=29.956444229969
F2=−13.2208430269052
F3=24.509258301827
D1=32.8947783569125
D2=22.1052216430875

$$S = C = ( \begin{matrix}- 1 & 100 \\ 0 & - 1\end{matrix} )$$

For the example, the total track T plus the camera lens length was reduced from 400 mm to 65 mm and the paraxial optical system matrix for the two systems were the same. This is a reduction in total system length of about 6:1 which is a significant improvement in terms of overall optical length, which is the objective. The final optical system layout is illustrated in FIG. 10, illustrating the simplified optical system for the compact ocular wavefront system having long working distance.

Some simple extensions to the method described above are: other global optimization algorithms could be used to solve for the lenses and axial separations. For example, genetic algorithms, or combination simulated annealing and genetic algorithms could be used.

True two- or three-dimensional ray tracing could be used in place of paraxial system matrices to solve for the lens surfaces, thicknesses, and axial separations.

A weighted error could be used in the optimization routine to give more importance to, for example, ray height (first row of system matrix) over ray angle (second row of system matrix). Rather than a prototype two-lens relay system, the prototype system matrix S could be given directly.

The working distance WD could be any useful value. It is not required to be equal to Fa.

The system matrices S and C could be taken from any two points along the optical system. It is not a requirement that they be taken from the first lens in the relay system to the sensor plane.

The method could be used to provide a compact relay lens for any optical system, the utility is not limited to use in an ocular wavefront system.

The three lenses could be combined to form a single cemented lens.

The method could be extended to four or more lenses. The method could be applied to surface powers. The same approach could be used to include the selection of one or more of the lenses from a catalog of discrete available lenses or surfaces or glasses, that is, any combination of continuous and discrete parameters.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A compact ocular wavefront system with a long working distance and utilizing a compact three-lens relay system that matches the optical characteristics of a longer two-lens relay system wherein said wavefront system provides a significantly reduced optical path versus the two-lens relay system without reducing overall optical performance.

2. The compact ocular wavefront system according to claim 1 where a wavefront sensor is placed next to a camera without an intermediate lens.

3. The compact ocular wavefront system according to claim 1 where an error expression of equation
$$E = {\sum }_{m = 0}^{1} {\sum }_{n = 0}^{1} {W}_{mn} \times {\vert {S}_{mn} - {C}_{mn} \vert}^{p}$$
is minimized using simulated annealing or some other global optimization scheme.

4. The compact ocular wavefront system of claim 3 where the error E is calculated using weights and corresponding elements from the S and C matrixes.

5. The compact ocular wavefront system of claim 3 where the weights Wmn allow individual terms in the system matrixes to receive more importance than others.

6. The compact ocular wavefront system of claim 3 where the parameter p allows to weight larger errors more or less than small errors.

7. The compact ocular wavefront system of claim 4 where the error expression of equation has weights assigned other than 1 to specify relative importance of different system matrix elements.

8. The compact ocular wavefront system of claim 1 where a working distance is a short distance.

9. The compact ocular wavefront system of claim 1 where a working distance is an arbitrary distance.
